(19) United States
(12) Patent Application Publication
(10) Pub. No.: US 2017/0281127 A1
(43) Pub. Date: Oct. 5, 2017

(54) ULTRASOUND PROBE

(71) Applicant: KONICA MINOLTA, INC., Tokyo (JP)

(72) Inventor: Michiyo HIRAYAMA, Kanagawa (JP)

(21) Appl. No.: 15/475,856

(22) Filed: Mar. 31, 2017

(30) Foreign Application Priority Data
Mar. 31, 2016 (JP) 2016-071081

(51) Int. Cl. A61B 8/00; A61B 8/12

(52) U.S. Cl. CPC A61B 8/4444; A61B 8/4483; A61B 8/461; A61B 8/12

(57) ABSTRACT

An ultrasound probe includes, on a surface of a casing, a center indication part configured to indicate an approximate center position of at least one side of an ultrasound transmission/reception side surface of the piezoelectric element, and the center indication part is formed such that two or more tangents intersect each other at at least a first intersection and a second intersection on a virtual center line extending in an ultrasound transmission/reception direction of the piezoelectric element and passing through the approximate center position, and that the tangents intersecting each other have gradients of opposite signs.

### CROSS REFERENCE TO RELATED APPLICATIONS

This application is entitled to and claims the benefit of Japanese Patent Application No. 2016-071081, filed on Mar. 31, 2016, the disclosure of which including the specification, drawings and abstract is incorporated herein by reference in its entirety.

### BACKGROUND OF THE INVENTION

### 1. Field of the Invention

The present invention relates to an ultrasound probe for ultrasound diagnosis, and particularly relates to an ultrasound probe for performing a paracentesis while observing an ultrasound image acquired by transmission/reception of ultrasound waves.

### 2. Description of Related Art

Ultrasound diagnostic apparatuses can, for example, acquire the shape, movement, and the like of tissues in the form of an ultrasound diagnosis image by simply bringing an ultrasound probe, which can be connected or can communicate with the ultrasound diagnostic apparatus, into contact with the body surface or by inserting the ultrasound probe into the body. Accordingly, ultrasound diagnostic apparatuses have a high level of safety, and can be repeatedly used for inspection.

In recent years, a biopsy in which a puncture needle is inserted to the patient's body as the subject to collect tissues and bodily fluid is known. In addition, in an anesthetic department, an intensive care unit, a pain clinic and the like, treatments using a puncture needle are performed. In such diagnoses or treatments, the operator such as a doctor inserts a puncture needle while confirming the position of the puncture needle with reference to an ultrasound image of body tissues acquired with the ultrasound diagnostic apparatus.

At this time, to reduce the burden of the patient and correctly perform puncture as much as possible, it is necessary to accurately determine the insertion position of the puncture needle on the patient's body surface, and the relative position of the insertion position of the puncture needle on the patient's body surface and the targeted position in the ultrasound image displayed on the monitor.

For example, Japanese Patent Application Laid-Open No. 2010-119484 discloses a technique in which a center indication part that indicates an approximate center position of the arranged transducer in the longitudinal direction is provided on the external surface of the casing of the ultrasound probe for the purpose of surely marking the scan center position of the ultrasound probe to the patient's body surface. It should be noted that this technique is not designed for insertion of the puncture needle to the patient's body from the marked position on the patient's body surface.

In addition, Japanese Registered Utility Model No. 3190119 discloses a technique in which a center indication line that indicates the center position of the arranged transducer in the longitudinal direction and the short direction is provided on the external surface of the casing of the ultrasound probe for the purpose of correctly performing a paracentesis, and the puncture needle is inserted to the patient's body from the position of the patient's body surface indicated by the center indication line.

Since the ultrasound probe disclosed in Japanese Patent Application Laid-Open No. 2010-119484 is designed for puncture through an adapter (fixing part 29 and holding part 25) for guiding the puncture needle as described above, the structure of the ultrasound probe is complicated, and consequently, operations such as mounting of an adapter is required to perform a paracentesis. Further, since puncture is performed through the adapter fixed to the ultrasound probe, the puncture angle of the needle is limited, and as a result the operator such as a doctor cannot freely perform operations, making it difficult to accurately perform the puncture.

In contrast, in an ultrasound probe such as the ultrasound probe disclosed in Japanese Registered Utility Model No. 3190119, the center indication line that indicates the scan center position of the ultrasound probe is provided, and the ultrasound probe is configured to insert the puncture needle into the patient's body from a position of the patient's body surface indicated by the center indication line. With this configuration, the ultrasound probe such as the ultrasound probe disclosed in Japanese Registered Utility Model No. 3190119, does not require the adapter or the like, and the operator such as a doctor can freely perform puncture. However, when the indication part that indicates the scan center position of the ultrasound probe has a straight-line shape as in the invention disclosed in Japanese Registered Utility Model No. 3190119, the directivity toward the scan center position of the ultrasound probe is not provided, and consequently the operator such as a doctor cannot correctly recognize the scan center position of the ultrasound probe.

In addition, when the center indication line has a large thickness relative to the puncture needle, the center position cannot be easily recognized, and the scan center position cannot be correctly indicated. Conversely, when the thickness of the center indication line is reduced so that the center position can be easily recognized, the center indication line cannot be easily visually-recognized.

As described above, disadvantageously, the conventional ultrasound probes cannot be freely operated by the operator such as a doctor, or, cannot correctly indicate the scan center position. In either case, error or shifting of the insertion position of the puncture needle on the patient's body surface occurs, and a paracentesis cannot be accurately performed, thus increasing the burden on the patient.

### SUMMARY OF THE INVENTION

An object of the present invention is to provide an ultrasound probe which can accurately perform a paracentesis without missing the insertion position of the puncture needle on the patient's body surface.

To achieve the abovementioned object, an ultrasound probe reflecting one aspect of the present invention includes: a piezoelectric element configured to transmit and receive ultrasound waves; and a casing configured to house the piezoelectric element. A center indication part configured to indicate an approximate center position of at least one side of an ultrasound transmission/reception side surface of the piezoelectric element is provided on a surface of the casing, the center indication part is formed such that two or more tangents intersect each other at at least a first intersection and a second intersection on a virtual center line extending in an ultrasound transmission/reception direction of the piezoelectric element and passing through the approximate center position, and that the tangents intersecting each other have gradients of opposite signs, the first intersection is located at a position on an ultrasound transmission/reception side of the piezoelectric element, the second intersection is located at a position on a side opposite to the ultrasound transmission/reception side of the piezoelectric element relative to the first intersection, the tangents intersecting at the first intersection form a projection toward the ultrasound transmission/reception side of the piezoelectric element, and the tangents intersecting at the second intersection form a projection toward the ultrasound transmission/reception side of the piezoelectric element or a recess with respect to the ultrasound transmission/reception side of the piezoelectric element.

### BRIEF DESCRIPTION OF DRAWINGS

FIG. 1 is a perspective view of an external appearance of an ultrasound diagnostic apparatus using an ultrasound probe;

FIG. 2 is a plan view illustrating an external appearance of the ultrasound probe;

FIG. 3 is a perspective view of an external appearance of the ultrasound probe;

FIG. 4 is a sectional view illustrating a main structure of the ultrasound probe;

FIG. 5 is an exploded view illustrating the main structure of the ultrasound probe;

FIG. 6 illustrates an arrowhead-like shape of a puncture mark;

FIG. 7A and FIG. 7B are explanatory diagrams for describing an effect for a case where an end of the puncture mark on an ultrasound transmission/reception side cannot be visually recognized;

FIG. 8A to FIG. 8D are explanatory diagrams for describing an effect for a case where the puncture mark is shifted or deformed;

FIG. 9 is an explanatory diagram for describing an arrow-feather-like shape of the puncture mark;

FIG. 10A and FIG. 10B are explanatory diagrams for describing a modification of the arrowhead-like shape of the puncture mark;

FIG. 11A to FIG. 11C are explanatory diagrams for describing a modification of another shape of the puncture mark;

FIG. 12 is a sectional view illustrating a main structure of an ultrasound probe of the modification; and

FIG. 13 is an exploded view illustrating the main structure of the ultrasound probe of the modification.

### DESCRIPTION OF THE PREFERRED EMBODIMENTS

An embodiment of the present invention will be described below with reference to the drawings.

### Ultrasound Diagnostic Apparatus

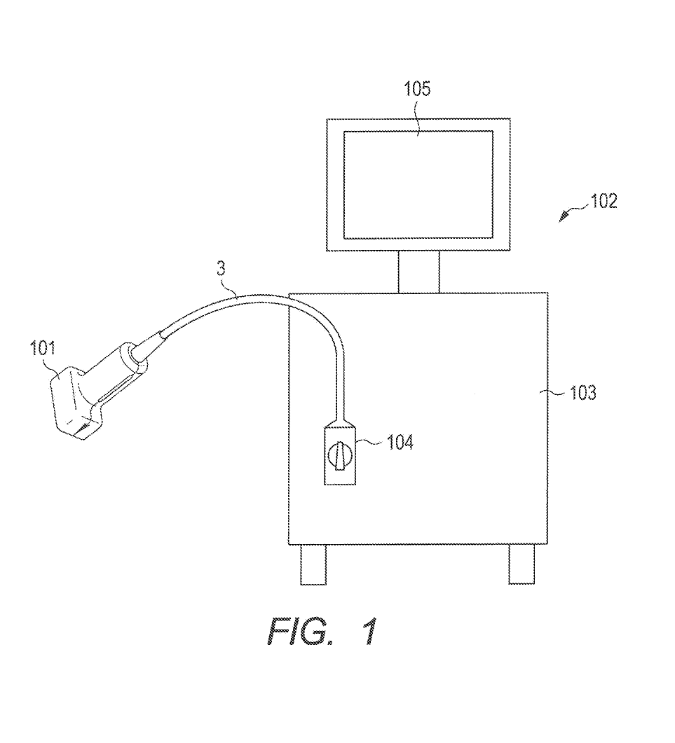

FIG. 1 is a perspective view of an external appearance of ultrasound diagnostic apparatus 102 using ultrasound probe 101 according to the present embodiment.

Ultrasound diagnostic apparatus 102 includes ultrasound diagnostic apparatus main body 103, connector part 104 and display 105.

Ultrasound probe 101 is connected with ultrasound diagnostic apparatus 102 through cable 3 connected with connector part 104.

An electric signal (transmission signal) from ultrasound diagnostic apparatus 102 is transmitted to a piezoelectric element part of ultrasound probe 101 through cable 3. It is to be noted that the piezoelectric element part will be described later. This transmission signal is converted to ultrasound waves at the piezoelectric element part, and transmitted into a living body. The transmitted ultrasound waves are reflected by tissues and the like in the living body, and a part of the reflection wave is received and converted by the piezoelectric element into an electric signal (received signal), and thereafter, transmitted to ultrasound diagnostic apparatus 102 through cable 3. The received signal is converted to image data at ultrasound diagnostic apparatus 102, and displayed on display 105.

The ultrasound probe is described below in detail.

### Ultrasound Probe

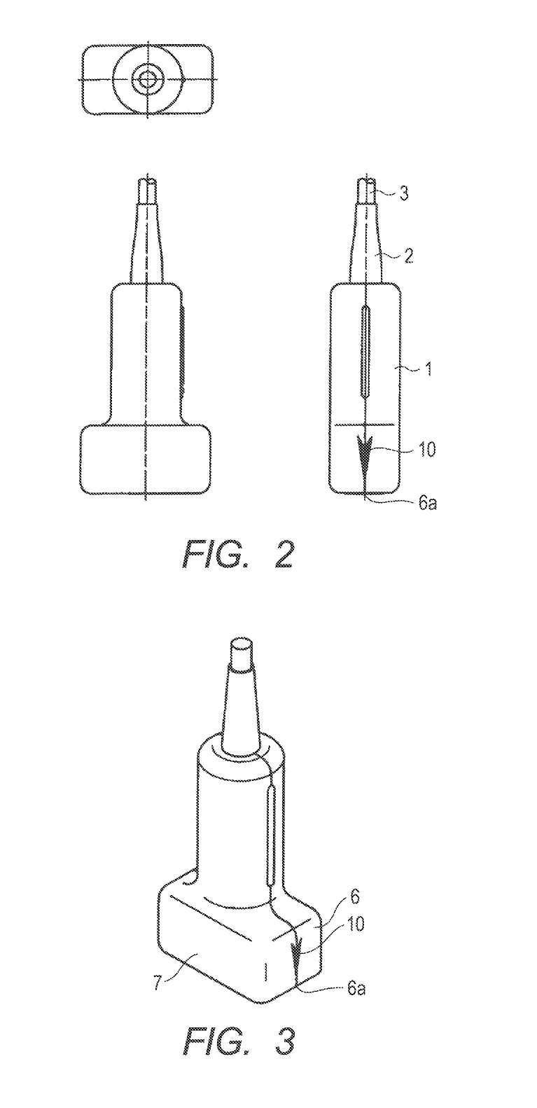

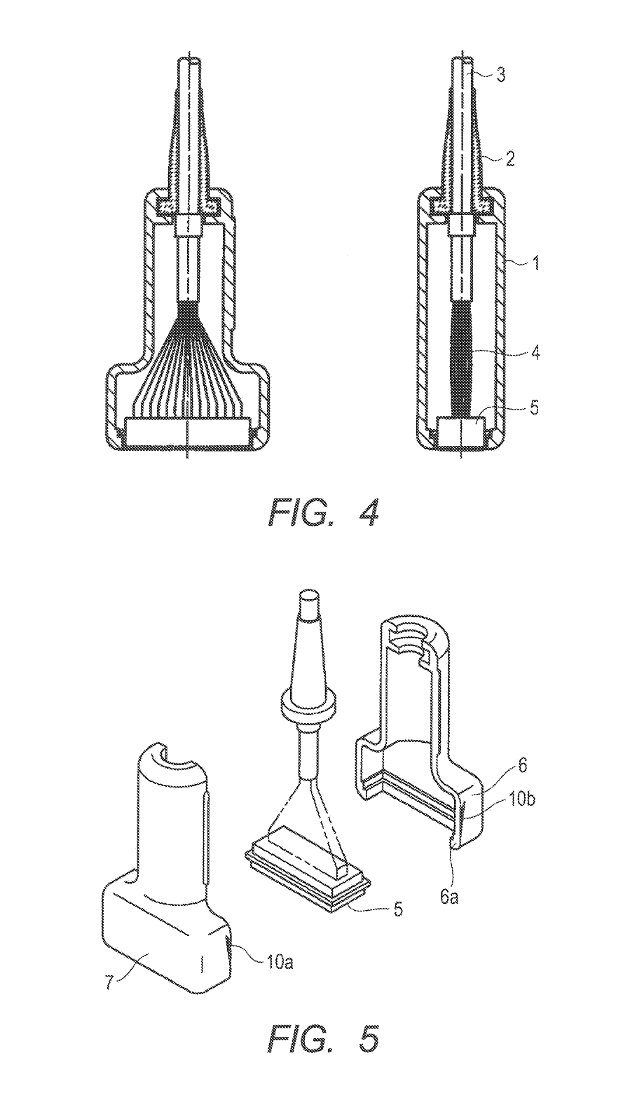

FIG. 2 and FIG. 3 are a plan view and a perspective view illustrating an example external appearance of ultrasound probe 101, respectively. FIG. 4 and FIG. 5 are a sectional view and an exploded view illustrating an example main structure of ultrasound probe 101, respectively.

As illustrated in FIG. 2 and FIG. 4, ultrasound probe 101 includes piezoelectric element part 5, electric signal line connection part 4 and casing 1, and is provided with cable 3 and cable bushing 2.

Piezoelectric element part 5 transmits and receives ultrasound waves. Piezoelectric element part 5 may be composed of one piezoelectric body (not illustrated). Alternatively, piezoelectric element part 5 may be composed of a plurality of piezoelectric bodies arranged in one direction such that the arrangement direction of the piezoelectric bodies is the longitudinal direction of piezoelectric element part 5 and a direction substantially perpendicular to the arrangement direction of piezoelectric bodies is the short direction of piezoelectric element part 5. Electric signal line connection part 4 is connected with piezoelectric element part 5 and cable 3, and transmits an electric signal from ultrasound diagnostic apparatus 102 or piezoelectric element part 5. Cable 3 is connected with ultrasound diagnostic apparatus main body 103. Cable bushing 2 prevents disconnection of cable 3 due to abrupt bending or the like.

As illustrated in FIG. 5, casing 1 can be separated into two parts which are combined so as to cover piezoelectric element part 5, electric signal line connection part 4, cable 3, cable bushing 2 and the like after the components are combined together. Casing 1 combined in this manner is joined and integrated with a silicone adhesive agent or the like such that it serves as a holding part designed to be held by the operator or the like.

Portions 10a and 10b which serve as a center indication part (hereinafter referred to also as “puncture mark”) that indicates the scan center position of ultrasound probe 101 are provided across joint surface 6a of the two parts of casing 1. When casing 1 is combined and integrated, portions 10a and 10b are combined together to form one puncture mark 10 as illustrated in FIG. 2 and FIG. 3. Details of the puncture mark will be described later.

While casing 1 has a configuration which can be separated into two parts as described above, casing 1 may be separated into three or more parts.

In addition, while the external appearance of ultrasound probe 101 illustrated in FIG. 1 to FIG. 5 is an external appearance of a linear scanning type ultrasound probe, the present invention is not limited to this. For example, it is also possible to employ a probe of convex scanning type, a probe of an electron sector scan type, or a probe of a body cavity insertion type which is inserted to the patient's body cavity when in use.

In addition, while ultrasound probe 101 is connected with ultrasound diagnostic apparatus 102 through cable 3, ultrasound probe 101 may be connected with ultrasound diagnostic apparatus 102 through radio communication without providing the cable.

### Puncture Mark

As described above, puncture mark 10 that indicates the center position of the short side of the ultrasound transmission/reception side surface of piezoelectric element part 5 is provided across joint surface 6a of the two parts of casing 1 as the center indication part that indicates the scan center position of ultrasound probe 101.

As illustrated in FIG. 5, when casing surface 7 of the longitudinal side of piezoelectric element part 5 is the front surface, joint surface 6a is formed on casing side surface 6 of the short side of piezoelectric element part 5 and substantially agrees with the center line of casing side surface 6 which is parallel to the ultrasound transmission/reception direction.

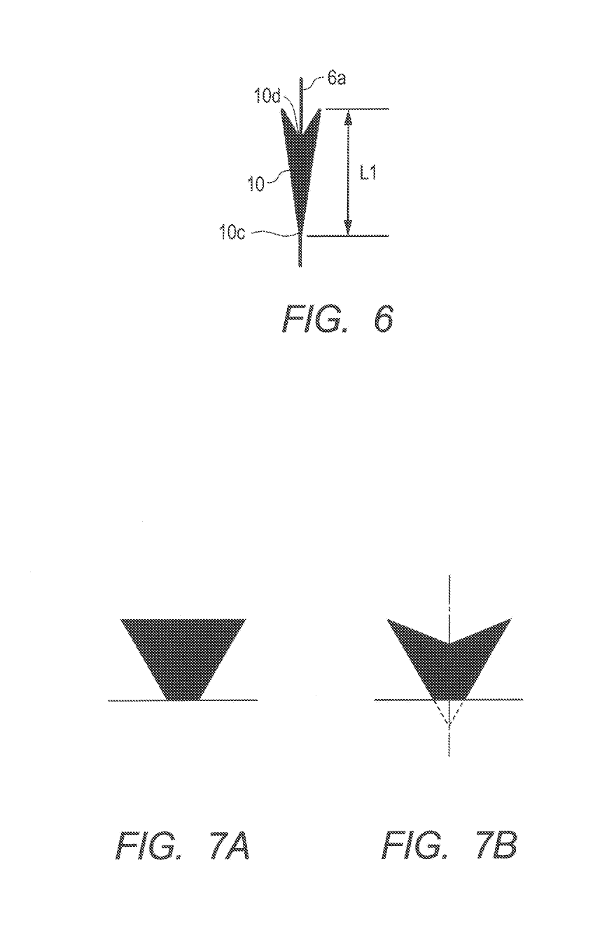

As illustrated in FIG. 6, puncture mark 10 has an arrowhead-like shape, and is formed such that end portion 10c indicates the center position of the short side of the ultrasound transmission/reception side surface of piezoelectric element part 5 (hereinafter referred to also as “scan center position”).

The arrowhead-like shape is composed such that the tangents to the shapes of the portions provided on respective two parts of casing 1 intersect at intersections A and B located on joint surface 6a.

Two tangents which intersect at intersection A on the ultrasound transmission/reception side of piezoelectric element part 5 have gradients of opposite signs, and in addition, the two tangents form an acute projection directed toward the ultrasound transmission/reception side of piezoelectric element part 5. With this configuration, the shape of the portion of puncture mark 10 where the tangents intersect each other at intersection A on the ultrasound transmission/reception side of piezoelectric element part 5 (hereinafter referred to as “lower portion”) naturally has a shape concentrating toward joint surface 6a which substantially coincides with the scan center position of ultrasound probe 101, and thus it is possible to indicate the direction toward the scan center position of ultrasound probe 101, and correctly indicate the scan center position.

In addition, when ultrasound diagnostic apparatus 102 is used for diagnosis or treatment, the operator such as a doctor in some situation may strongly presses ultrasound probe 101 against the patient's body surface to obtain more favorable signals. In this case, the patient's body surface is recessed by being pressed, and a part of the surface of the ultrasound probe making contact with the patient's body surface is put into the recessed portion of the patient's body surface and cannot be visually recognized.

In this case, even when a puncture needle is directly inserted into the patient's body from a position on the patient's body surface corresponding to the indication part that indicates the scan center position without interposing an adapter or the like by use of the ultrasound probe disclosed in Japanese Patent Application Laid-Open No. 2010-119484 for example, the indication part has an inverted triangular shape, and consequently the vertex of the inverted triangular shape on the living body side, which should indicate the scan center position, cannot be visually recognized. At this time, since the portion of the indication part exposed from the recessed portion of the patient's body surface is substantially trapezoid as illustrated in FIG. 7A, the operator such as a doctor cannot correctly recognize the scan center position of the ultrasound probe.

However, with puncture mark 10 according to the present embodiment, even in the case where ultrasound probe 101 is put into the patient's body and end portion 10c and cannot be visually recognized when in use, the operator such as a doctor can estimate the tangents to portions 10a and 10b of puncture mark 10 based on the direction toward the scan center position indicated by the shape of the lower portion of puncture mark 10, and can determine the position of intersection A of the scan center position of the ultrasound probe based on the tangents. Thus, the operator can correctly recognize the scan center position of the ultrasound probe.

Moreover, the arrowhead-like shape includes a shape of a portion (hereinafter referred to as “upper portion”) where the tangents intersect each other at intersection B located at a position on the side opposite to the ultrasound transmission/reception side of piezoelectric element part 5. The two tangents which intersect each other at intersection B also have gradients of opposite signs, and thus puncture mark 10 can indicate the scan center position of ultrasound probe 101 not only with end portion 10c of the lower portion of the patient's body surface side, but also with end portion 10d of the upper portion on the side opposite to the patient's body surface side in a paracentesis using ultrasound diagnostic apparatus 102. Therefore, when ultrasound probe 101 is put into the patient's body and end portion 10c cannot be visually recognized, end portion 10d of the upper portion is remote from the patient's body surface, and exposed from the patient's body surface without being put into the patient's body. Thus, end portion 10d of the upper portion can surely indicate the scan center position of ultrasound probe 101, and can help the operator such as a doctor to estimate intersection A based on the shape of the lower portion of puncture mark 10 and determine the scan center position of ultrasound probe 101 as described above. That is, as illustrated in FIG. 7B, the operator such as a doctor can further accurately recognize the scan center position of ultrasound probe 101 by determining the position of intersection B based on end portion 10d of the upper portion, and estimating a virtual center line that passes through intersection A and intersection B.

Further, in the case where the center indication part that indicates the scan center position of the ultrasound probe is provided across the joint surface of two parts of the casing of the ultrasound probe as in the present embodiment, the combined center indication part is shifted or deformed due to fitting shift of the casing, or size error or positional error of the center indication part of the two parts of the casing.

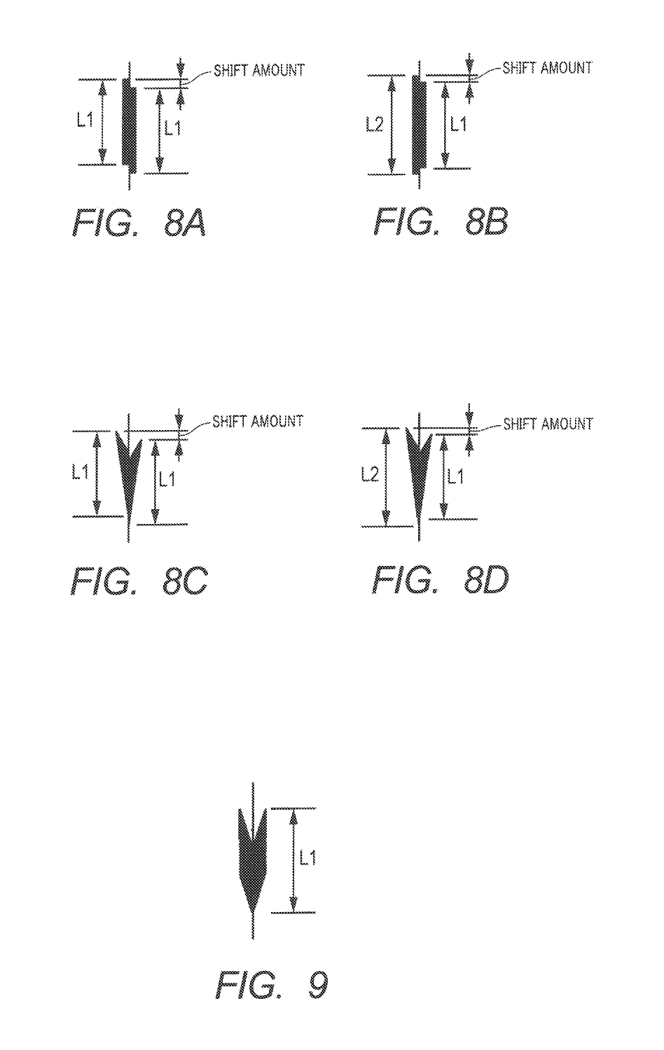

For example, in the case where the indication part that indicates the scan center position of the ultrasound probe has a straight-line shape as in the invention disclosed in Japanese Registered Utility Model No. 3190119, the combined indication part is shifted or deformed when two portions each having length L1 of the two parts of the casing are combined while being vertically shifted due to fitting shift by a shift amount as illustrated in FIG. 8A, or when two portions respectively having length L1 and length L2 resulting from a positional error or a size error are combined together as illustrated in FIG. 8B. In either case, the operator such as a doctor cannot correctly recognize the scan center position of the ultrasound probe.

However, with puncture mark 10 according to the present embodiment in which the lower portion of the arrowhead-like shape has a shape concentrating toward joint surface 6a and indicates the direction toward the scan center position of ultrasound probe 101 as described above, the operator such as a doctor is not disturbed even when end portion 10c is mistakenly shifted or deformed as illustrated in FIG. 8C and FIG. 8D, and can correctly recognize the scan center position of ultrasound probe 101.

While puncture mark 10 according to the present embodiment has an arrowhead-like shape, the shape of puncture mark 10 is not limited to the arrowhead-like shape as long as the tangents to the shape which intersect each other at joint surface 6a have gradients of opposite signs, and the tangents which intersect each other at the intersection on the ultrasound transmission/reception side of piezoelectric element part 5 form a projection directed toward the ultrasound transmission/reception side of piezoelectric element part 5.

In addition, to ensure exposure of end portion 10d of the upper portion of puncture mark 10 when end portion 10c on the ultrasound transmission/reception side of piezoelectric element part 5 cannot be visually recognized, it is preferable to provide a certain distance between intersection A on the ultrasound transmission/reception side and intersection B on the side opposite to intersection A of piezoelectric element part 5. For example, puncture mark 10 may have an arrow-feather-like shape as illustrated in FIG. 9.

In addition, while puncture mark 10 according to the present embodiment is formed such that the tangents to the shape of the lower portion form an acute projection directed toward the ultrasound transmission/reception side of piezoelectric element part 5, the projection may not have an acute angle. For example, the tangents to the shapes of the lower portion of puncture mark 10 may form a projection having an obtuse angle directed toward the ultrasound transmission/reception side of piezoelectric element part 5.

In addition, while puncture mark 10 according to the present embodiment is formed such that the tangents to the shapes of the upper portion form a projection directed toward the ultrasound transmission/reception side of piezoelectric element part 5, puncture mark 10 may form a recess with respect to the ultrasound transmission/reception side of piezoelectric element part 5. For example, even when puncture mark 10 is formed in a rhombus-like shape or a hexagonal-like shape as illustrated in FIG. 11A and FIG. 11B, it is possible to recognize the scan center position of ultrasound probe 101 with end portion 10d of the upper portion, and it is possible to accurately perform a paracentesis without missing the insertion position of the puncture needle on the patient's body surface even when end portion 10c of the lower portion cannot be visually recognized.

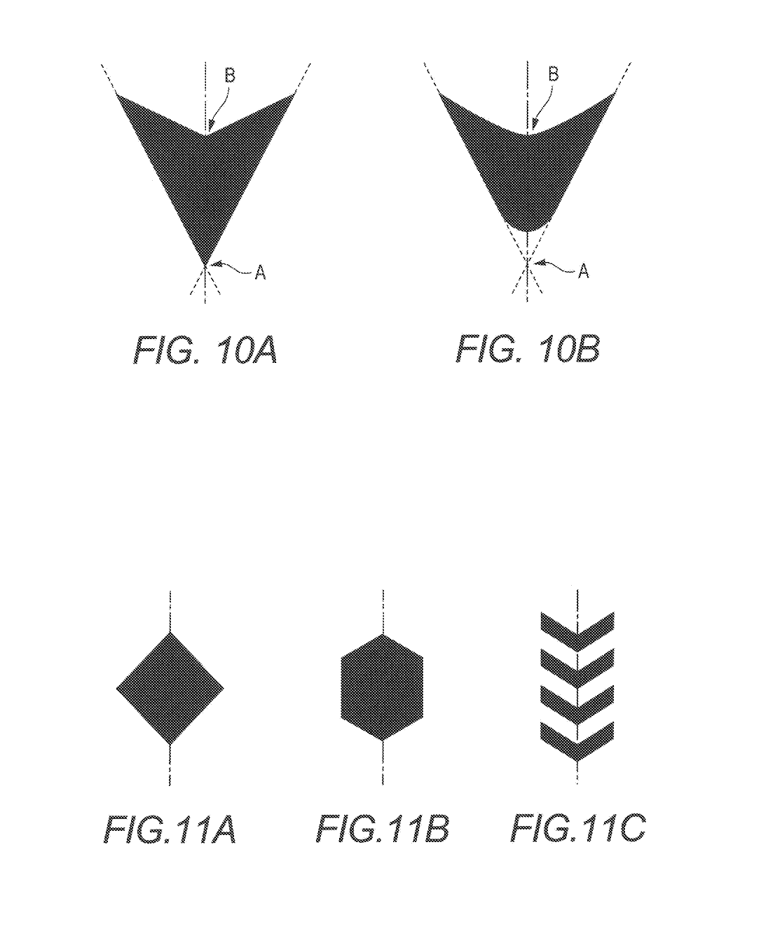

In addition, while puncture mark 10 according to the present embodiment is formed such that the shapes of the upper portion and the lower portion and joint surface 6a directly intersect each other (that is, the outline of the shape and the tangent thereof is the same line) as illustrated in FIG. 10A, puncture mark 10 may be formed such that the outlines of the shapes of the upper portion and the lower portion and joint surface 6a do not directly intersect each other but only the tangents intersect each other at the intersection on joint surface 6a as illustrated in FIG. 10B. For example, as illustrated in FIG. 10B, the front end portions of the shapes of the upper portion and the lower portion of puncture mark 10 may be rounded, or, may be formed in a straight line shape which is substantially parallel to the ultrasound transmission/reception side surface of piezoelectric element part 5, in place of the rounded portion.

In addition, while the tangents to the shapes and joint surface 6a intersect each other at intersections A and B in puncture mark 10 according to the present embodiment, the tangents to the shapes and joint surface 6a may be intersect each other at two or more intersections as long as the tangents to the shape intersecting each other at joint surface 6a have gradients of opposite signs, and the tangents intersecting each other at the intersection on the ultrasound transmission/reception side of piezoelectric element part 5 form a projection directed toward the ultrasound transmission/reception side of piezoelectric element part 5. For example, puncture mark 10 may be formed as illustrated in FIG. 11C, or may be formed by a combination of different shapes.

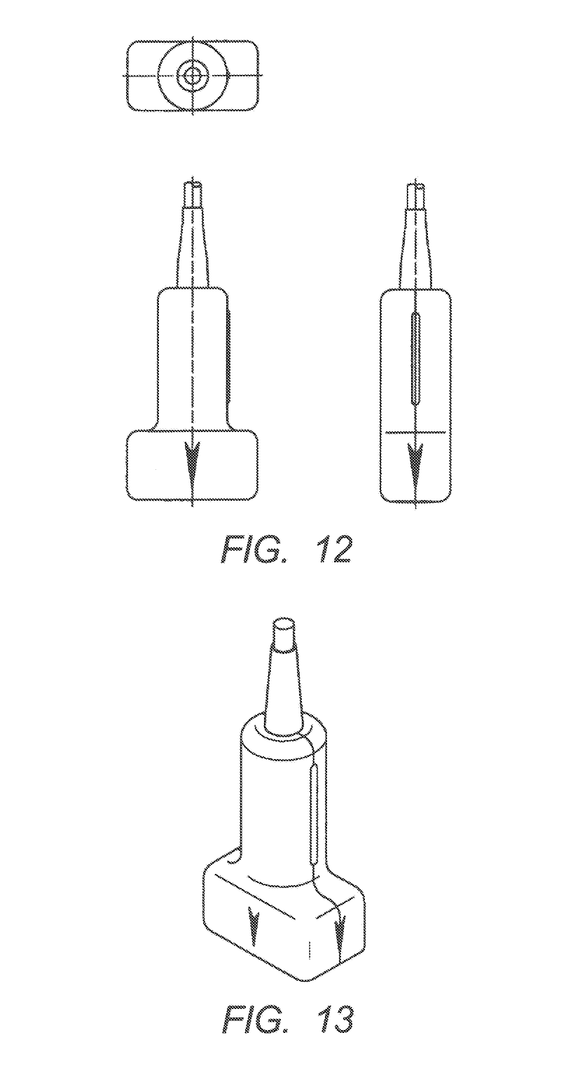

In addition, when the puncture needle is inserted from the short side of piezoelectric element part 5 in a paracentesis, substantially the entire puncture needle can be shown in the ultrasound image, and the end of the puncture needle is shown throughout the operation, thus making it easy to observe the puncture needle. In view of this, in the present embodiment, the indication part (puncture mark 10) for indicating the center position of the short side of the ultrasound transmission/reception side surface of piezoelectric element part 5 is provided on casing side surface 6 of the short side of piezoelectric element part 5 such that the puncture needle can be inserted from the short side. Alternatively, or in addition thereto, an indication part for indicating the center position of the long side of the ultrasound transmission/reception side surface of piezoelectric element part 5 may be provided on the longitudinal side surface of the ultrasound probe as illustrated in FIG. 12 and FIG. 13.

Desirably, puncture mark 10 is formed in a highly-chemically resistive manner such as irregularities formed by molding, laser marking, or printing. In addition, puncture mark 10 may have multiple colors.

### Effect of the Embodiment

As described above, ultrasound probe 101 can directly insert the puncture needle into the patient's body from a position of the patient's body surface corresponding to puncture mark 10 without using an adapter or the like for guiding the puncture needle in a paracentesis, and thus the operator such as a doctor can freely perform the operation.

In addition, puncture mark 10 is formed such that the tangents to the portions 10a and 10b provided on respective two parts of casing 1 intersect at intersections A and B located on joint surface 6a which substantially coincides with the center line which is parallel to the ultrasound transmission/reception direction of casing side surface 6, and that the tangents intersecting each other have gradients of opposite signs. Moreover, the tangents which intersect at intersection A on the ultrasound transmission/reception side of piezoelectric element part 5 form a projection directed toward the ultrasound transmission/reception side of piezoelectric element part 5. With this configuration, the direction toward the scan center position of ultrasound probe 101 is indicated, and thus the scan center position of ultrasound probe 101 can be correctly indicated.

In addition, even in the case where end portion 10c on the ultrasound transmission/reception side of piezoelectric element part 5 cannot be visually recognized when in use, puncture mark 10 surely indicates the direction toward the scan center position of ultrasound probe 101 with end portion 10c of the lower portion intersecting at intersection A on the ultrasound transmission/reception side of piezoelectric element part 5, and can surely indicate the scan center position of ultrasound probe 101 with end portion 10d of the upper portion intersecting at intersection B on the side opposite to the ultrasound transmission/reception side of piezoelectric element part 5. Thus, the operator such as a doctor can correctly recognize the scan center position of ultrasound probe 101, and can accurately perform a paracentesis without missing the insertion position of the puncture needle on the patient's body surface.

Further, even when portions 10a and 10b provided on respective two parts of casing are shifted or deformed due to factors such as fitting shift and manufacturing error, puncture mark 10 indicates the direction toward the scan center position of ultrasound probe 101, and therefore the shifting or the deformation does not interfere with recognition of the scan center position. As a result, the operator such as a doctor can correctly recognize the scan center position of ultrasound probe 101, and can accurately perform a paracentesis without missing the insertion position of the puncture needle on the patient's body surface.

What is claimed is:

1. An ultrasound probe comprising:
a piezoelectric element configured to transmit and receive ultrasound waves; and
a casing configured to house the piezoelectric element, wherein
a center indication part configured to indicate an approximate center position of at least one side of an ultrasound transmission/reception side surface of the piezoelectric element is provided on a surface of the casing,
the center indication part is formed such that two or more tangents intersect each other at at least a first intersection and a second intersection on a virtual center line extending in an ultrasound transmission/reception direction of the piezoelectric element and passing through the approximate center position, and that the tangents intersecting each other have gradients of opposite signs,
the first intersection is located at a position on an ultrasound transmission/reception side of the piezoelectric element,
the second intersection is located at a position on a side opposite to the ultrasound transmission/reception side of the piezoelectric element relative to the first intersection,
the tangents intersecting at the first intersection form a projection toward the ultrasound transmission/reception side of the piezoelectric element, and
the tangents intersecting at the second intersection form a projection toward the ultrasound transmission/reception side of the piezoelectric element or a recess with respect to the ultrasound transmission/reception side of the piezoelectric element.

2. The ultrasound probe according to claim 1, wherein the center indication part includes, at an end thereof on the ultrasound transmission/reception side of the piezoelectric element, or at another end thereof on a side opposite to the ultrasound transmission/reception side of the piezoelectric element, a round portion, or a straight line portion which is substantially parallel to the ultrasound transmission/reception side surface of the piezoelectric element.

3. The ultrasound probe according to claim 1, wherein
an outline of the center indication part intersects the virtual center line at at least the first intersection and the second intersection,
the outline intersecting at the first intersection forms a projection toward the ultrasound transmission/reception side of the piezoelectric element, and
the outline intersecting at the second intersection forms a projection toward the ultrasound transmission/reception side of the piezoelectric element or a recess with respect to the ultrasound transmission/reception side of the piezoelectric element.

4. The ultrasound probe according to claim 1, wherein
the piezoelectric element is composed of a plurality of piezoelectric bodies disposed in a first direction, and
the center indication part is formed on a side surface of the casing which is substantially perpendicular to the first direction.

5. The ultrasound probe according to claim 1, wherein
the casing is composed of at least two parts, and
the virtual center line substantially coincides with a joint surface of the parts.

6. The ultrasound probe according to claim 1, wherein the center indication part has an arrowhead shape, an arrow feather shape, or a rhombus shape.

7. The ultrasound probe according to claim 1, wherein the center indication part is composed of an irregularity formed by printing, laser marking, or molding.

8. The ultrasound probe according to claim 1, wherein the center indication part has one or more colors which are different from a color of the casing.

9. The ultrasound probe according to claim 8, wherein the center indication part has one or more colors which are stronger than the color of the casing.

10. An ultrasound diagnostic apparatus comprising the ultrasound probe according to claim 1.
